(19) United States
(12) Patent Application Publication
(10) Pub. No.: US 2004/0147564 A1
(43) Pub. Date: Jul. 29, 2004

(54) Combinations of glimepiride and the thiazolidinedione for treatment of diabetes

(76) Inventors: Vinay U. Rao, Aurangabad (IN); Dilip Gopalkrishna Saoji, Aurangabad (IN); Sunil Anantrao Mirajkar, Aurangabad (IN); Prashant Prabhakarrao Deshmukh, Aurangabad (IN); Harshal Prabhakar Bhagwatwar, Aurangabad (IN); Manjusha Malhotra, New Delhi (IN); Milind C. Shukla, Aurangabad (IN); Noel John De Souza, Mumbai (IN)

Correspondence Address: KENYON & KENYON, ONE BROADWAY, NEW YORK, NY, 10004, US

(21) Appl. No.: 10/641,965

(22) Filed: Aug. 16, 2003

(30) Foreign Application Priority Data
Jan. 29, 2003 (IN) 106/MUM/2003

(51) Int. Cl. A61K 31/4439; A61K 31/426; A61K 31/175

(52) U.S. Cl. 514/342; 514/369; 514/592

(57) ABSTRACT

A unit-dose pharmaceutical composition for the treatment of non-insulin dependent diabetes mellitus includes a combination of glimepiride and a thiazolidinedione insulin sensitizer, providing for the simultaneous release of each drug at rates substantially similar to those obtained with the separate administration of immediate release dosage forms of glimepiride and the thiazolidinedione. In addition, processes for the preparation of such combination unit-dose compositions and the use of such compositions for improving glycemic control are described.

### FIELD OF THE INVENTION

The present invention relates to a stable unit-dose combination for the simultaneous delivery of glimepiride and a thiazolidinedione insulin sensitizer for the treatment of non-insulin dependent diabetes mellitus and improvement of glycemic control. The present invention also relates to processes for the preparation of such combination unit-dose formulations and the use of such combinations in the treatment of diabetes.

### BACKGROUND OF THE INVENTION

Diabetes mellitus is a progressive metabolic disorder in human beings characterized by hyperglycemia and insulin resistance. It is often associated with other disorders such as obesity, hypertension, hyperlipidemia, as well as complications such as cardiovascular disease, retinopathy, and nephropathy. Diabetes can be classified into two major classes: (1) Insulin dependent diabetes mellitus (IDDM or Type I diabetes), wherein the patients lack beta cells in the pancreas, and such patients are treated with insulin; and (2) Non-insulin dependent diabetes mellitus (NIDDM or Type II diabetes), wherein the patients possess beta cells with impaired insulin secretion function.

Initially, diet and exercise is the mainstay of treatment of type II diabetes. However, these are followed by administration of oral hypoglycemic agents. Based on their mechanism of action, the antidiabetic agents are mainly classified as follows:

1. Biguamides, represented principally by metformin, phenformin and buformin, act by decreasing hepatic glucose production and intestinal absorption of glucose, while enhancing peripheral glucose uptake;

2. Sulfonylureas (also known as insulin secretagogues), represented principally by glipizide, glimepiride, glyburide, glibomuride, glisoxepide, gliclazide acetohexamide, chlorpropamide, tolazamide, and tolbutamide, among others, act by stimulating the release of insulin from the beta cells of the pancreas;

3. Thiazolidinediones, represented principally by the class of glitazones including, for example, rosiglitazone, troglitazone and pioglitazone, among others, act by increasing the sensitivity of insulin receptors in the body and diminish or eliminate the need for exogenous insulin; and

4. Alpha-glucosidase inhibitors, represented principally by acarbose and miglitol, among others, act by delaying absorption of dietary carbohydrates.

As described above, these agents have a different mechanism of action, unique to each class of drugs. These hypoglycemic agents, in monotherapy, are used as a first-line treatment in diabetic patients. However, after an initial positive response, these antidiabetic agents become ineffective as a secondary failure sets in. Besides a qualitative and/or quantitative deficiency of insulin secretion, the patients develop insulin resistance. Sulfonylureas target one aspect of hyperglycemia by augmenting insulin secretion from the beta cells, while thiazolidinediones act by increasing the sensitivity of insulin available in the blood stream. The different yet complementary mechanisms of action suggest a therapeutically viable rationale for use of combinations of such agents in targeting hyperglycemia.

The simultaneous use of two or more biologically active agents from the aforementioned different classes to achieve a synergistic effect has previously been demonstrated. For example, three-way combinations were described in U.S. Patent Application Publication No. 20020016287 A1, which described the combination of a biguamide, an insulin sensitizer and an insulin secretagogue for the treatment of NIDDM. U.S. Pat. Nos. 6,166,043 and 6,172,090 claim a method for reducing the amounts and side effects of active components administered to a diabetic patient, which comprises administering a therapeutically effective amount of an insulin sensitivity enhancer in combination with a biguamide.

Sulfonylureas lower blood glucose levels acutely by stimulating the release of insulin from the pancreas, an effect dependent upon functioning beta cells in the pancreatic islets. They bind to sulfonylurea receptors on the beta cell plasma membrane, causing closure of ATP-sensitive potassium channels leading to depolarization of the cell membrane. This in turn opens voltage-gated calcium channels, allowing influx of calcium ions and subsequent secretion of insulin.

Sulfonylureas stimulate insulin secretion but cannot enhance insulin sensitivity, which is the primary requirement for treating insulin resistance that is a most common feature characterizing the pathogenesis of type II diabetes. The thiazolidinedione class of antidiabetic agents, represented by glitazones, does not incite insulin secretion, but improves glycemic control by improving insulin sensitivity. Glitazones are highly selective and potent agonists for the peroxisome proliferator-activated receptor-gamma (PPARγ). Activation of PPARγ nuclear receptors regulates the transcription of insulin responsive genes involved in the control of glucose production, transport, and utilization.

In addition, PPARγ-responsive genes also participate in the regulation of fatty acid metabolism. The antidiabetic activity of glitazones has been demonstrated in type II diabetes in which hyperglycemia and/or impaired glucose tolerance is a consequence of insulin resistance in target tissues.

The use of combinations of sulfonylureas and glitazones have been described in the art. For example, U.S. Pat. Nos. 6,150,383 and 6,329,404 describe the preparation and use of unit-dose combination compositions of glitazones with secretagogues for the treatment of diabetes. The use of a combination unit-dose composition containing pioglitazone and glibenclamide was also shown to be synergistic in lowering of plasma glucose over either of the compounds administered alone to fatty male Wistar rats. In addition, U.S. Patent Application Publication No. 20020147226 A1, described the two-way combination of a sulfonylurea (an insulin secretagogue) and a glitazone (an insulin sensitizer).

A combination of a sulfonylurea antidiabetic agent and rosiglitazone has been disclosed in U.S. Pat. No. 5,972,973 for the treatment of NIDDM in humans, with defined ranges of the two actives comprising 3-250 mg of sulfonylurea, and 5-50 mg of rosiglitazone. Furthermore, U.S. Pat. No. 5,859,037 discloses a composition comprising 3-250 mg of a sulfonylurea and 100-1000 mg of a glitazone to obtain a therapeutic effect for the treatment of diabetes.

The prior art defines an optimum therapeutic range for the oral administration of a combination of drugs belonging to the class of sulfonylureas and thiazolidinediones. This allows a range of daily doses, based on increasing the number of tablets taken per day, for the treatment of the ailment with drugs such as glipizide, having a short elimination half-life of about 2-3 hours. However, a single administration of a glitazone, for example, rosiglitazone or pioglitazone, activates the insulin receptors for an extended period and may thus be administered as a single dose without there being a need to maintain the plasma concentration of this drug. Likewise, use of a sulfonylurea that possesses a long terminal half-life eliminates the need for multiple medications of the drug and could be administered once-daily at a conventional dose. Drugs such as, for example, glyburide (terminal half-life of 10 hours) and glimepiride (terminal half-life of more than 5 hours), are suitable examples (See Physician's Desk Reference, 2002).

However, there is no availability in clinical practice of such combinations for the simultaneous immediate delivery of a glitazone and a sulfonylurea, all in one physically and chemically stable dosage form for ready once-a-day administration. A need for such a dosage form exists. The availability of a dosage form that can provide therapeutic levels of a sulfonylurea and a thiazolidinedione from the same unit-dose composition would be extremely constructive in clinical practice for glycemic control in the treatment of NIDDM. Such a product would improve the treatment of NIDDM through significantly enhanced patient compliance because of ease of administration and a reduced frequency of dosing. There is also the possibility of a significant reduction in the doses of the drug substances used in combination because of the synergistic action, resulting in a possible reduction in toxicity.

Thiazolidinediones and sulfonylureas are primarily insulin sensitizers and secretagogues, respectively, with proven efficacy in lowering glucose levels in the treatment of type II diabetes. Although long-acting sulfonylureas reduce glycosylated hemoglobin A_{1c}, (HbA_{1c}) levels by 0.8 to 2.0% and fasting plasma glucose (FPG) concentrations by 60 to 70 mg per dL (3.3 to 3.9 mmol per L), their use remains limited, particularly in an elderly population. Renal or hepatic insufficiency, in geriatric patients, causes elevated drug levels and also diminishes gluconeogenic capacity, both of which increase the risk of serious hypoglycemic reactions. Elderly, debilitated or malnourished patients, and those with adrenal or pituitary insufficiency, are particularly susceptible to the hypoglycemic action of such glucose-lowering drugs.

Furthermore, the risk of hypoglycemia may be increased with combination therapy. However, the use of glitazones in combination with glimepiride alleviates such a risk. The main site of action of glimepiride is the pancreatic beta cell, where the drug initiates insulin secretion by binding to the sulfonylurea receptor (SUR), but to a different part of the SUR than glyburide and other long-acting sulfonylureas. It provides a less marked stimulation of insulin secretion in proportion to its effect on lowering plasma glucose, due to a potent extrapancreatic effect, mainly in adipocytes. This results in fewer hypoglycemic episodes associated with glimepiride compared to glyburide and other long-acting sulfonylureas. This makes glimepiride a suitable sulfonylurea for use in the elderly where it is safe, efficacious and well tolerated, showing no significant effect on the cardiac or renal function of the patient (See Sinha et al., Annals of Long-Term Care: Clinical Care and Aging (2001) 9(6):23).

Glimepiride, chemically known as 1-[[p-[2-(3-ethyl-4-methyl-2-oxo-3-pyrroline-1-carboxamido) ethyl]phenyl]sulfonyl]-3-(trans-4-methylcyclohexyl)urea, has been described in U.S. Pat. No. 4,379,785, which is incorporated herein by reference. Glimepiride is a long-acting sulfonylurea having an elimination half-life of about 5-8 hours at steady state after oral administration, which increases with higher dose. It lowers blood glucose levels and reduces absolute HbA_{1c} levels by 1-2%. It has a marked insulin secretory effect, both in vitro and in vivo, increasing plasma insulin levels by as much as 50% in patients with type 2 diabetes (See Langtry et al., Drugs (1998) 55(4):563).

As aforementioned, the use of combination of thiazolidinediones and short acting sulfonylureas is described in the U.S. Pat. Nos. 5,972,973 and 5,859,037, but a sulfonylurea having long terminal half-life, particularly glimepiride, in combination with an appropriate thiazolidinedione is unknown previously in the art.

It would be desirable to provide a combination therapy of glimepiride and a glitazone having a synergistic effect on glucose control. Both of these agents act by different but complementary mechanisms. It would be desirable to add a thiazolidinedione agent to concurrent glimepiride treatment and provide a balance of stimulated release of insulin, while ameliorating insulin resistance. It would be advantageous to provide such a combination and provide a level of glycemic control unattainable by either medication alone.

Although the use of a combination of a thiazolidinedione and a sulfonylurea has been described in the art, the art does not describe the mode of preparation of compositions of such a combination that can be used for the therapeutic advantage to the benefit of the patient and clinician. Specifically, the use of a unit-dose composition comprising a glitazone (such as rosiglitazone or pioglitazone) and a sulfonylurea (such as glimepiride), which is also physically and chemically stable and releases both the antidiabetic agents simultaneously at a rate similar to that compared to the individually marketed products, such as for example, glimepiride from Amaryl™ (Aventis) and pioglitazone from G-Tase™ (Unichem) or rosiglitazone from Avandia™ (Smith Kline Beecham), has not been described.

Combinations of biologically active agents are especially difficult to formulate because of the inherent differences in physicochemical properties, the possible drug-drug interactions between the drugs, and the ingredients used for formulation of the combination composition. Furthermore, the oral combination products should be adaptable so that the extent of release, the release rates and profiles from the combination product are comparable to the two drugs dosed concurrently from separate formulations. This is a particularly challenging task for the pharmaceutical formulation scientist, especially in the development of a combination product because of issues such as the uniformity of content of the low dose drugs in the matrix, solubility characteristics and the amounts of excipients that can be used to formulate such a dosage form.

Therefore, a need exists in the art for a chemically and physically stable dosage form which can be administered once-a-day and which can provide therapeutic levels of glimepiride and a thiazolidinedione, for example, pioglitazone or rosiglitazone, from the same unit-dose composition in a pattern similar to each of the separate products available commercially. Such a dosage form would be extremely beneficial in clinical practice for glycemic control in the treatment of NIDDM for patients poorly controlled on either glimepiride or any thiazolidinedione alone.

There is also a need in the art for such a dosage form resulting in a bioequivalent product, which would provide similar in vivo release profiles for the two drugs when compared with each of the marketed products administered individually.

There is a further need in the art for such a once-a-day combination composition having a lower incidence of between-meal hypoglycemic events when compared with other once-a-day combinations, thus resulting in enhanced patient compliance. Also, such a once-a-day combination composition would provide enhanced efficacy through an increase in basal insulin levels because of the prolonged action of glimepiride, which would be further enhanced by the insulin sensitization provided by a thiazolidinedione.

### SUMMARY OF THE INVENTION

It is therefore an object of the present invention to provide a chemically and physically stable pharmaceutical composition for oral administration comprising a combination of pharmaceutically effective amounts of glimepiride and a thiazolidinedione.

It is a further object of the present invention to provide a pharmaceutical composition that provides an immediate release of glimepiride and a thiazolidinedione, simultaneously.

It is also an object of the present invention to provide a pharmaceutical composition that provides simultaneous release of glimepiride and a thiazolidinedione at rates substantially similar to those obtained with separate administration of an immediate release composition of glimepiride and an immediate release composition of a thiazolidinedione.

It is another object of the present invention to provide a pharmaceutical composition for a combination of glimepiride and a thiazolidinedione wherein the glimepiride has a mean particle size of less than about 30 microns and a particle size distribution such that at least 90% of the particles are less than about 75 microns.

It is yet another object of the present invention to provide a method of use of this combination for the treatment of diabetes and its associated maladies.

In keeping with these objectives, the present invention relates to a pharmaceutical composition for a combination of glimepiride and a thiazolidinedione or a pharmaceutically acceptable salt thereof wherein the composition provides a simultaneous release of each drug at rates substantially similar to those obtained with separate administration of immediate release dosage forms of glimepiride and a thiazolidinedione.

Glitazones, such as for example, pioglitazone and rosiglitazone, have low solubility characteristics. Rosiglitazone is readily soluble in a buffered aqueous solution with a pH of 2.3. Its solubility decreases with increasing pH in the physiological range (See Physician's Desk Reference, 2002). However, glimepiride is practically insoluble in water (100 μg/ml or less at 25° C.) and also hydrophobic in nature. The bioavailability of such a drug depends on its rate of solution upon administration. The hydrophobicity of this drug poses the key formulation impediment as it results in poor dissolution. A quick disintegration of the combination product provides dissolution rates of glimepiride unsuitably low due to the inherent solubility properties of the drug substance itself. It has now been found through extensive in-vitro testing that the reduced dissolution rates and thus bioavailability are related to the particle size and the particle size distribution of the glimepiride. The micronization of glimepiride improves the dissolution characteristics of the drug from the combination product and thus improves the bioavailability. However, it has been found that particles that were too small resulted in high dissolution rates of glimepiride and hence, higher blood levels with consequent risk of hypoglycaemia. Larger particles, however, do not dissolve sufficiently rapidly to give comparable dissolution profiles with the commercially available separate products.

It is therefore necessary to have a closely defined particle size distribution of the glimepiride in the combination product with a thiazolidinedione, such as a glitazone. The selection of a specific particle size fraction of glimepiride enables the production of a combination product exhibiting a glimepiride dissolution profile comparable to that obtained with the individual co-administration of a glitazone and glimepiride, and thus ensures the required equivalent bioavailability.

The usual starting dose of glimepiride at initiation of therapy is 1 to 2 mg followed by a maintenance dose of 1 to 4 mg once daily. Being a low dose drug, another challenge in formulating the dosage form of glimepiride as a combination product is to ensure homogeneity and thus good drug content uniformity. Furthermore, glimepiride is inherently very potent and thus the correct dose needs to be administered to the patient. It is extremely important during the formulation of solid oral dosage forms to avoid segregation of the powder blend and to maintain content uniformity of such low dose drugs. Uniformity of drug content is a serious limitation, and if not strictly assured, there could be a possibility of the desired therapeutic effect not being achieved through an underdose. On the other hand, incidents of hypoglycemia may occur due to overdose.

To achieve content uniformity of low dose drugs, methods, such as for example, the solution method or powder dilution method, are conventionally used for formulating the dosage forms. For the former, a solution of a drug dissolved in a proper solvent is uniformly dispersed in the excipient and then this dispersion is used for further processing to provide the dosage form. For the latter, geometric mixing is adopted, wherein the drug is first compounded with a small portion of the excipient followed by mixing with a slightly larger portion of the excipient and so on, until the desired mix is obtained which is then further processed to provide the dosage form.

However, the solution method is not suitable for hydrophobic drugs, such as for example, glimepiride, due to their low aqueous solubility. A drug in solid form when in contact with a solvent may change its crystal form, for example, a hydrate may change into a solvate and thus change the effective moiety. Furthermore, when the solution containing the drug is dispersed with the excipients for the preparation of the desired dosage form, the removal of solvent results in precipitation of the drug. The particle size of the drug may change and may affect the stability of the drug. This may result in the change in its physico-chemical characteristics and thus its biopharmaceutical behavior.

In addition, it is difficult to produce drug preparations having excellent drug content uniformity using the powder dilution method especially when a potent, low dose drug substance is involved. In particular, uniform mixing of aggregates of the drug with excipients is not attained by simple mixing and thus a homogenizing step such as mixture pulverization is required. Hence, for obtaining uniformity of drug content, complex manufacturing procedures are required.

It has now been found that the need for complex manufacturing procedures for attaining content uniformity can be avoided using micronized glimepiride with commercially available excipients, particularly lactose. Without wishing to be bound by any particular theory, it is postulated that one explanation for the achievement of content uniformity is that micronization of glimepiride develops a surface charge on the fine drug particles, which when blended with excipient particles of large size, adhere electrostatically to the larger excipient particles thus aiding in the prevention of segregation of the powder blend. This high binding affinity and low demixing potential of the micronized glimepiride helps in attaining enhanced homogeneity resulting in good content uniformity.

Thus, in one embodiment of the present invention, a pharmaceutical composition for oral administration comprises a combination of a pharmaceutically effective amount of glimepiride and a pharmaceutically effective amount of a thiazolidinedione or a pharmaceutically acceptable salt thereof, wherein the glimepiride has a mean particle size of less than about 30 microns and a particle size distribution such that at least 90% of the particles are less than about 75 microns. A further embodiment of the present invention comprises this same pharmaceutical composition wherein the composition provides a simultaneous release of each drug at rates substantially similar to those obtained with separate administration of immediate release dosage forms of glimepiride and a thiazolidinedione.

The present invention also includes antidiabetic combination compositions, and processes for the preparation of such compositions, comprising combinations of glimepiride and a thiazolidinedione for the treatment of NIDDM for improving glycemic control.

In one embodiment of the invention, a composition comprising glimepiride and a thiazolidinedione, all in the same dosage form, can be prepared in the form of, for example, pellets, beads, granules, tablets or capsules.

Another embodiment of the present invention includes a pharmaceutical composition in the form of a bilayered dosage form in a unit-dose combination for the simultaneous delivery of glimepiride and a thiazolidinedione to provide a synergistic effect for the treatment of diabetes. The term “bilayered” as used herein is meant to encompass solid dosage forms such as tablet formulations where there are two separate drug layers, one on top of the other with only one surface in mutual contact. These may be prepared by compressing additional granulation on a previously compressed granulation, or alternatively by feeding previously compressed tablets into a machine and compressing another granulation layer around the preformed tablets. The term “bilayered” also includes formulations where one drug component is coated onto the second drug component, which may be in the form of, for example, tablets, capsules, granules, pellets or beads.

### BRIEF DESCRIPTION OF THE DRAWINGS

FIG. 1 shows the comparative dissolution profile of pioglitazone HCl from G-Tase™ (Unichem) 15 mg tablets, and from an embodiment of the unit-dose combination composition of the present invention comprising glimepiride 1 mg+pioglitazone HCl 15 mg tablets (Example 1).

FIG. 2 shows the comparative dissolution profile of glimepiride from Amaryl™ (Aventis) 1 mg tablets, and from an embodiment of the unit-dose combination composition of the present invention comprising glimepiride 1 mg+pioglitazone HCl 15 mg tablets (Example 1).

FIG. 3 shows the comparative dissolution profile of pioglitazone HCl from G-Tase™ (Unichem) 15 mg tablets, and from an embodiment of the unit-dose combination composition of the present invention comprising glimepiride 2 mg+pioglitazone HCl 15 mg tablets (Example 2).

FIG. 4 shows the comparative dissolution profile of glimepiride from Amaryl™ (Aventis) 2 mg tablets, and from an embodiment of the unit-dose combination composition of the present invention comprising glimepiride 2 mg+pioglitazone HCl 15 mg tablets (Example 2).

FIG. 5 shows the comparative dissolution profile of pioglitazone HCl from G-Tase™ (Unichem) 30 mg tablets, and from an embodiment of the unit-dose combination composition of the present invention comprising glimepiride 1 mg+pioglitazone HCl 30 mg tablets (Example 3).

FIG. 6 shows the comparative dissolution profile of glimepiride from Amaryl™ (Aventis) 1 mg tablets, and from an embodiment of the unit-dose combination composition of the present invention comprising glimepiride 1 mg+pioglitazone HCl 30 mg tablets (Example 3).

FIG. 7 shows the comparative dissolution profile of pioglitazone HCl from G-Tase™ (Unichem) 30 mg tablets, and from an embodiment of the unit-dose combination composition of the present invention comprising glimepiride 2 mg+pioglitazone HCl 30 mg tablets (Example 4).

FIG. 8 shows the comparative dissolution profile of glimepiride from Amaryl™ (Aventis) 2 mg tablets, and from an embodiment of the unit-dose combination composition of the present invention comprising glimepiride 2 mg+pioglitazone HCl 30 mg tablets (Example 4).

### DETAILED DESCRIPTION

An embodiment of the present invention includes an immediate release unit-dose composition for the once-a-day administration of glimepiride, along with a thiazolidinedione, such as for example, pioglitazone or rosiglitazone, which is physically and chemically stable, and releases each drug from the unit-dose composition at a rate similar to that of the individually marketed products such as, for example, glimepiride from Amaryl™ (Aventis), and pioglitazone from G-Tase™ (Unichem) or rosiglitazone from Avandia™ (Smith Kline Beecham). Another embodiment of the present invention further includes processes for the preparation of such compositions and their use for the control of hyperglycemia in the treatment of NIDDM and associated conditions.

In accordance with the invention, any salts, solvates, hydrates, polymorphs, complexes and such other products of glimepiride, may also be employed.

According to an embodiment of the invention, the particle size of the glimepiride is such that the mean particle size is less than about 30 microns, and the particle size distribution is such that at least 90% of the glimepiride particles are less than about 75 microns.

Preferably, the particle size of the glimepiride is such that the mean particle size is about 25 microns, and the particle size distribution is such that at least 90% of the glimepiride particles are less than about 60 microns.

According to another embodiment, the micronized glimepiride is evenly distributed over different sized sieve fractions of thiazolidinedione and other excipients resulting in a homogenous mixture. Furthermore, during processing constituent segregation is minimized resulting in a powder blend with good content uniformity.

The glimepiride of the invention is preferably present in an amount of from about 0.2% to about 8% by weight, more preferably from about 0.5% to about 3.5% by weight of the total composition.

The thiazolidinediones that could be used in accordance with the unit-dose combinations of the present invention include, but are not limited to, pioglitazone and rosiglitazone, and other medicinally active and pharmaceutically acceptable forms from the glitazone class of compounds, including their salts, solvates, hydrates, polymorphs, complexes and such other products.

Preferably, the pharmaceutical composition of the invention includes each drug, glimepiride and the thiazolidinedione, in an amount that is typically administered for a given period of time. This amount includes a pharmaceutically effective amount of the drug, which is an amount large enough to significantly positively modify the condition to be treated, but small enough to avoid serious side-effects (at a reasonable benefit/risk ratio), within the scope of sound medical judgement.

The thiazolidinedione of the invention is preferably present in an amount of from about 0.5% to about 45% by weight of the total composition.

In those embodiments of the invention where the thiazolidinedione is pioglitazone or a pharmaceutically acceptable salt of pioglitazone, it is preferably present in an amount of from about 4% to about 45% by weight, more preferably from about 12% to about 38% by weight, of the total composition.

In embodiments of the present invention where the thiazolidinedione is rosiglitazone or a pharmaceutically acceptable salt of rosiglitazone, it is preferably present in an amount of from about 0.5% to about 10% by weight, more preferably from about 1.5% to about 7% by weight, of the total composition.

According to an embodiment of the present invention, the pharmaceutical composition may also contain other conventional pharmaceutically acceptable excipients known in the art of formulation development. The present invention is not to be construed as being limited to any particular excipient or class of pharmaceutical excipients. In addition, any adjuvants employed are preferably selected such that there is no interaction that would substantially reduce the pharmaceutical efficacy of the composition of the present invention. Pharmaceutical excipients used are preferably of high purity and low toxicity to render them suitable for administration. The choice of these excipients and the amounts to be used is considered to be within the purview of one of ordinary skill in the art and would depend on the type of individual dosage form. Such excipients include, but are not limited to, binding agents, diluents, disintegrants, glidants, wetting agents, lubricating agents, pigments, dyes and the like, and are known to persons skilled in the art of developing and manufacturing pharmaceutical solid oral dosage forms.

Examples of the binding agents which may be used in the present invention include, but are not limited to, hydroxypropyl cellulose, hydroxyethyl cellulose, sodium carboxymethyl cellulose, polyvinylpyrrolidone, alginates, starches, polysaccharides, and/or mixtures thereof. When present, the binder agent preferably comprises about 1% to about 10% by weight of the total composition.

According to another embodiment of the present invention, the pharmaceutical composition may also contain one or more of a water-soluble and/or a water dispersible diluent. Examples of water soluble diluents that may be used in the present invention include, but are not limited to, lactose, calcium sulphate, calcium phosphates, mannitol, dextrates, dextrin, dextrose, sucrose and the like. Water dispersible diluents, which refer to water insoluble pharmaceutical excipients that disperse readily in water, include but are not limited to, cellulose based excipients such as microcrystalline cellulose, powdered cellulose, starches such as corn starch, pregelatinized starch, clays or clay minerals such as kaolin, bentonite, attapulgite, and the like. When present, the diluent preferably comprises about 15% to about 85% by weight of the total composition.

Suitable disintegrating agents that may be used in the present invention include but are not limited to starch, croscarmellose sodium, sodium starch glycolate, crospovidone, cross-linked carboxymethyl starch, magnesium aluminium silicate, polyacrylin potassium, and the like. When present, the disintegrating agent is preferably present in an amount of from about 1% to about 10% by weight of the total composition.

In accordance with the present invention, other additives, such as for example, surface active agents, bioavailability modifiers, modifiers for absorption and the like, may also be present in the composition, and when present, they preferably comprise about 0.5% to about 5% by weight of the total composition. According to a further embodiment of the invention, the composition may optionally include a buffering agent, complexing agent and the like.

According to the present invention, the pharmaceutical composition may be prepared in a variety of forms including, but not limited to, pellets, beads, granules, tablets or capsules.

In those embodiments of the present invention wherein the pharmaceutical composition is in the form of a solid dosage form, it may contain in addition to the above ingredients, pharmaceutical-grade magnesium stearate or stearic acid and the like as a glidant, talc and the like as an anti-adherent, silicon dioxide or hydrogenated vegetable oil and the like as a lubricant, and ferric oxide and the like as a coloring agent.

According to an embodiment of the present invention, the pharmaceutical composition may be optionally coated with a rapidly dissolving water-soluble film coat. The examples of water-soluble polymers include, but are not limited to, hydroxypropyl methylcellulose, hydroxypropyl cellulose, and the like. When such an optional coat is present, the solid unit dosage form in accordance with the present invention is preferably coated to a weight build-up of about 1% to about 10% by weight, more preferably from about 1% to about 4% by weight of the total composition.

In those embodiments of the present invention wherein the pharmaceutical composition is in the form of a capsule dosage form, the capsule shell may be, for example, of a hard gelatin or a soft gelatin type.

In accordance with an embodiment of the present invention, glimepiride is subjected to micronization using any milling apparatus whereby it is reduced to a very fine powder due to attrition of the particles by collisions between particles and between particles and machine surfaces. The micronization may be advantageously carried out in an accelerated air-jet mill wherein collision of drug particles with each other under a high pressure stream of air causes reduction of particle size and increases the specific surface area of the material, manifold. The period of milling may vary depending on the size of the mill, the velocity of the air, the type of feed material and the quantity of feed material. The effects of these variables are well known in the art, and the present invention may be processed over a range of these variables. However, the comminution operation is preferably carried out until the powder obtained is such that the mean particle size is less than 30 microns and the particle size distribution is such that at least 90% of the particles are less than about 75 microns.

In an embodiment of the method of the present invention, the pharmaceutical composition is prepared by blending drugs, such as glimepiride and a thiazolidinedione, with pharmaceutically acceptable excipients, such as inert diluents and the like. The blend is directly compressed into tablets or may be filled into capsules. In another embodiment for the process of preparing the composition of the present invention, the drugs are blended with excipients, and the blend is roll compacted and then sized to obtain granules. The granules may be filled into capsules or compressed into tablets. In yet another embodiment for the process of preparing the composition of the present invention, the drugs are blended with above-mentioned excipients, and the blend is granulated with a solution of a binder. The granules so obtained are dried, sized and may be filled into capsules or compressed into tablets.

In those embodiments of the present invention wherein the foregoing composition is, for example, a tablet dosage form, the glimepiride and the rosiglitazone or pioglitazone may be intimately dispersed in the same tablet matrix or a bilayered tablet may be prepared which encompass formulations where two separate layers are prepared by the compression of individual granules containing the two active agents, or one active agent is present in a coating which is formed on a core containing the second active agent.

In those embodiments of the present invention wherein the foregoing composition is, for example, a capsule dosage form, it may contain a simple blend of the two active agents with the addition of suitable excipients or non-pareil sugar seeds and the like coated with the active ingredients filled into the capsule shells. Of course, the use of a tablet of one active and a powder or granules of the other active, both filled into a capsule is well within the spirit or scope of this invention as well.

In those embodiments of the present invention wherein the foregoing composition is in the form of spherical pellets or beads, such dosage forms may be produced by known techniques of extrusion and spheronisation techniques or techniques based on high shear granulation or fludized bed techniques, for example. Furthermore, embodiments of the invention including single unit pellets can be produced on an industrial scale using lozenge and troches cutting machines.

According to an embodiment of the invention, the combination unit-dose compositions could contain, for example, from about 0.5 to about 10 mg of glimepiride, and from about 5 to about 50 mg of pioglitazone or about 0.5 to about 10 mg of rosiglitazone. Preferably, these combination unit-dose compositions could contain from about 1 to about 4 mg of glimepiride, and from about 15 to about 45 mg of pioglitazone or about 2 to about 8 mg of rosiglitazone.

As would be understood by one of ordinary skill in the art, for any particular subject being treated, e.g., a mammal, specific dosage regimens should be adjusted according to the individual need, and such individual dosage regimens are within the scope of the invention. It is further to be understood that the dosages set forth herein are examples only and they do not to any extent limit the scope of the practice of the present invention. Preferably, the compositions of the invention are administered to the patients in need thereof either immediately before or after a meal in the morning or at night. That is, the preferred times for administering the compositions of the present invention are anytime within approximately 5-60 minutes before or after a meal in the morning, such as breakfast, or an evening meal, such as dinner.

The following examples further illustrate this invention and are not to be construed as limiting the scope of the invention. The examples are illustrative only and are to be read in conjunction with the description above to provide a further understanding of the present invention, and an exemplary outline of the process for preparing the composition of the invention.

### EXAMPLE 1

This example illustrates the present invention in the form of unit-dose tablet containing glimepiride and pioglitazone as the active ingredients. The pharmaceutical composition of this example is given below in Table 1.

TABLE 1

| | Weight of the |
|---|---|
| Ingredients | composition (mg) |
| Pioglitazone hydrochloride | 16.6 |
| (equivalent to pioglitazone | |
| 15 mg) | |
| Glimepiride | 1.0 |
| Microcrystalline cellulose | 15.0 |
| Lactose monohydrate | 76.9 |
| Polyvinylpyrrolidone (K-30) | 3.5 |
| Crosslinked | 5.0 |
| polyvinylpyrrolidone | |
| Purified talc | 0.5 |
| Magnesium stearate | 1.0 |
| Colloidal silicon dioxide | 0.5 |

Coating Compostion:

| | Weight of the coating |
|---|---|
| Ingredients | composition (mg) |
| Hydroxypropyl | 2.0 |
| methylcellulose | |
| Polyethyleneglycol 400 | 0.3 |
| Titanium dioxide | 0.5 |
| Coloring agent (FD&C Blue | 0.5 |
| No. 1) | |
| Isopropyl alcohol | q.s. |
| Methylene chloride | q.s. |

In this example micronized glimepiride (particle size of about 25 μ) was mixed with a part of lactose monohydrate in geometric proportion. Pioglitazone hydrochloride was added to the above blend and mixed well. Microcrystalline cellulose and remaining part of lactose monohydrate were further added to the above mixture and granulated with a dispersion of polyvinylpyrrolidone in isopropyl alcohol. The dried granules were sifted through a 850 μm mesh (British Standard Sieve (BSS) No. 18). The sized granules were blended with crosslinked polyvinylpyrrolidone, magnesium stearate, talc and colloidal silicon dioxide prior to compression into tablets.

Hydroxypropyl methylcellulose and polyethylene glycol were dissolved in a mixture of methylene chloride and isopropyl alcohol. Titanium dioxide was then dispersed in the above solution and homogenized. The tablets were film coated with this coating solution to a desired weight gain.

Characterization Studies

Content Uniformity of the Novel Unit-Dose Combination Composition:

The coated tablets prepared above according to example 1 were tested for the uniformity of content of pioglitazone and glimepiride. Ten (10) sample tablets (denoted below in Table 2 as CU-1 through CU-10) demonstrated an assay of glimepiride of 101.21% of label claim (1 mg per tablet) with a uniformity of content of 101.04%±5.48% and an assay of pioglitazone of 100.16% of label claim (15 mg per tablet) with a uniformity of content of 101.88%±5.43%. The content uniformity of each tablet studied in this example is given below in Table 2.

TABLE 2

| Tablets for | | |
|---|---|---|
| Content | Glimepiride | Pioglitazone |
| Uniformity | 1 mg (%) | 15 mg (%) |
| CU-1 | 97.44 | 93.07 |
| CU-2 | 98.00 | 98.54 |
| CU-3 | 98.45 | 96.28 |
| CU-4 | 100.00 | 98.23 |
| CU-5 | 97.91 | 100.38 |
| CU-6 | 108.67 | 107.50 |
| CU-7 | 108.75 | 108.32 |
| CU-8 | 93.76 | 100.98 |
| CU-9 | 108.49 | 107.26 |
| CU-10 | 98.96 | 108.23 |
| | Mean = 101.04 | Mean = 101.88 |
| | SD = 5.48 | SD = 5.43 |
| | RSD = 5.42 | RSD = 5.42 |

The data demonstrates good uniformity of content of the glimepiride and pioglitazone hydrochloride in the tablets.

Dissolution Profile of the Novel Unit-Dose Combination Composition:

The dissolution profile of the unit-dose combination composition as prepared above according to example 1, was compared to the profiles of each of the individually marketed products contained therein.

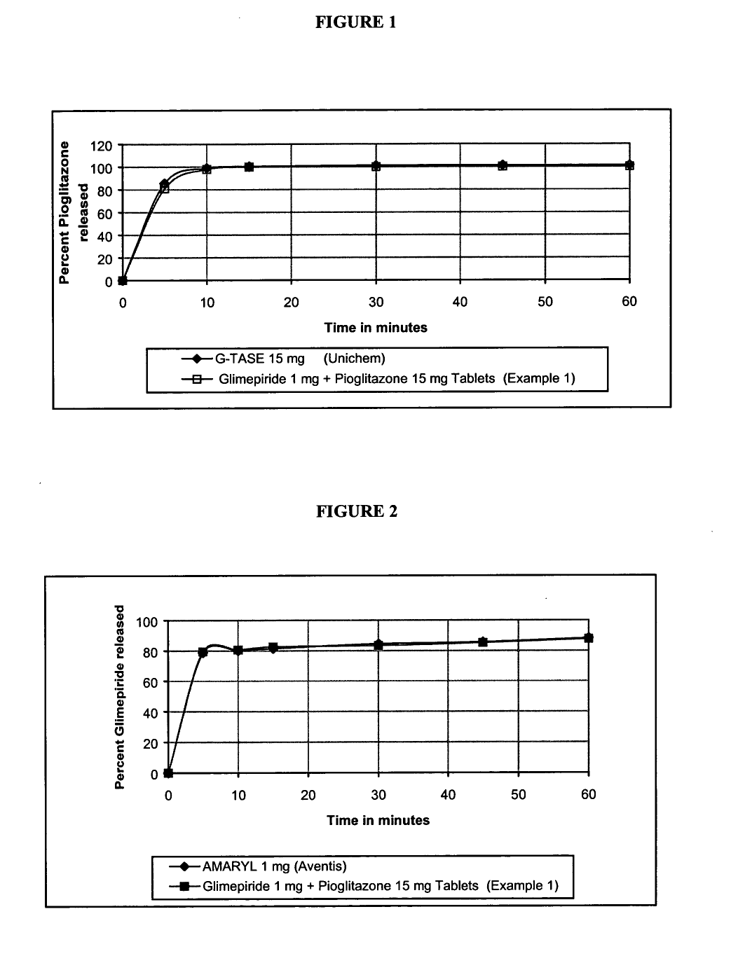

The dissolution profile for pioglitazone from the unit-dose combination composition of example 1 above was compared to that obtained for pioglitazone (15 mg) from its individually marketed form (G-TASE™ (UNICHEM)). The dissolution results are recorded in Table 3 and the profiles are given in FIG. 1. As shown in FIG. 1, which plots the data from Table 3, the unit-dose combination composition of example 1 releases the pioglitazone at a substantially similar rate to that of the individually marketed product.

TABLE 3

Cumulative percent pioglitazone released

| | | Glimepiride 1 mg + |
|---|---|---|
| Time | G-Tase ™ 15 mg | pioglitazone 15 mg tablets |
| (Minutes) | (Unichem) | (Example 1) |
| 5 | 85.60 | 80.87 |
| 10 | 98.91 | 98.00 |
| 15 | 100.32 | 100.00 |
| 30 | 101.11 | 100.00 |
| 45 | 101.23 | 100.08 |
| 60 | 101.23 | 100.23 |

Furthermore, the dissolution profile for glimepiride from the unit-dose combination composition of Example 1 was compared to that of glimepiride from its individually marketed form (AMARYL™ (AVENTIS)). The dissolution results are recorded in Table 4 and the profiles are given in FIG. 2. As shown in FIG. 2, which plots the data from Table 4, the unit-dose combination composition of Example 1 releases the glimepiride at substantially the same rate as the individually marketed product.

TABLE 4

Cumulative percent glimepiride released

| | | Glimepiride 1 mg + |
|---|---|---|
| Time | Amaryl ™ 1 mg | pioglitazone 15 mg tablets |
| (Minutes) | (Aventis) | (Example 1) |
| 5 | 78.80 | 79.52 |
| 10 | 80.22 | 80.63 |
| 15 | 81.43 | 82.55 |
| 30 | 84.52 | 83.42 |
| 45 | 85.68 | 85.18 |
| 60 | 88.33 | 87.72 |

Stability of the Novel Unit-Dose Combination Composition:

The unit-dose combination composition of Example 1, was also subjected to accelerated stability testing by storing the product in controlled temperature chambers at 40° C. and 75% relative humidity for three (3) months. There was no change in the physical properties of the tablets such as color and shape. Furthermore, there was no chemical degradation of the active agents as seen from the assay values at three months, which were 100.68% and 99.11% of the respective label claims of pioglitazone and glimepiride.

Thus, this example 1, which employs pioglitazone hydrochloride and glimepiride as the active agents of the composition, shows that the novel unit-dose combination composition of the present invention is physically and chemically stable under accelerated stability testing conditions of elevated temperature and humidity.

### EXAMPLE 2

This example illustrates the present invention in the form of unit-dose tablet containing pioglitazone (15 mg) and a higher dose of glimepiride (2 mg) than was used in Example 1. The pharmaceutical composition of this example is given below in Table 5.

TABLE 5

| | Weight of the |
|---|---|
| Ingredients | composition (mg) |
| Pioglitazone hydrochloride | 16.6 |
| (equivalent to pioglitazone 15 | |
| mg) | |
| Glimepiride | 2.00 |
| Microcrystalline cellulose | 15.0 |
| Lactose monohydrate | 76.0 |
| Polyvinylpyrrolidone (K-30) | 3.5 |
| Crosslinked | 5.0 |
| polyvinylpyrrolidone | |
| Purified talc | 0.5 |
| Magnesium stearate | 1.2 |
| Colloidal silicon dioxide | 0.5 |

Coating Compostion:

| | Weight of the coating |
|---|---|
| Ingredients | composition (mg) |
| Hydroxypropyl | 2.0 |
| methylcellulose | |
| Polyethyleneglycol 400 | 0.3 |
| Titanium dioxide | 0.5 |
| Coloring agent (FD&C Blue | 0.5 |
| No. 1) | |
| Isopropyl alcohol | q.s. |
| Methylene chloride | q.s. |

In this example the tablets were prepared and studied for content uniformity, dissolution and stability as described previously in example 1.

Characterization Studies

Content Uniformity of the Novel Unit-Dose Combination Composition:

The coated tablets of Example 2 were tested for the uniformity of content of pioglitazone and glimepiride. Ten (10) sample tablets (denoted below in Table 6 as CU-1 through CU-10) demonstrated an assay of glimepiride of 100.22% of label claim (2 mg per tablet) with a uniformity of content of 99.44%±3.73% and an assay of pioglitazone of 100.63% of label claim (15 mg per tablet) with a uniformity of content of 100.37%±3.56%. The content uniformity of each tablet studied in this example is given below in Table 6.

TABLE 6

| Tablets for | | |
|---|---|---|
| Content | Glimepiride | Pioglitazone |
| Uniformity | 2 mg (%) | 15 mg-(%) |
| CU-1 | 98.11 | 97.64 |
| CU-2 | 98.43 | 102.75 |
| CU-3 | 97.57 | 97.32 |
| CU-4 | 99.39 | 96.44 |
| CU-5 | 94.67 | 104.67 |
| CU-6 | 96.37 | 99.87 |
| CU-7 | 104.23 | 98.71 |
| CU-8 | 98.51 | 106.98 |
| CU-9 | 99.72 | 101.77 |
| CU-10 | 107.36 | 97.41 |
| | Mean = 99.44 | Mean = 100.37 |
| | SD = 3.73 | SD = 3.56 |
| | RSD = 3.75 | RSD = 3.54 |

The data demonstrates good uniformity of content of pioglitazone hydrochloride and for higher dose of glimepiride in the tablets.

Dissolution Profile of the Novel Unit-Dose Combination Composition:

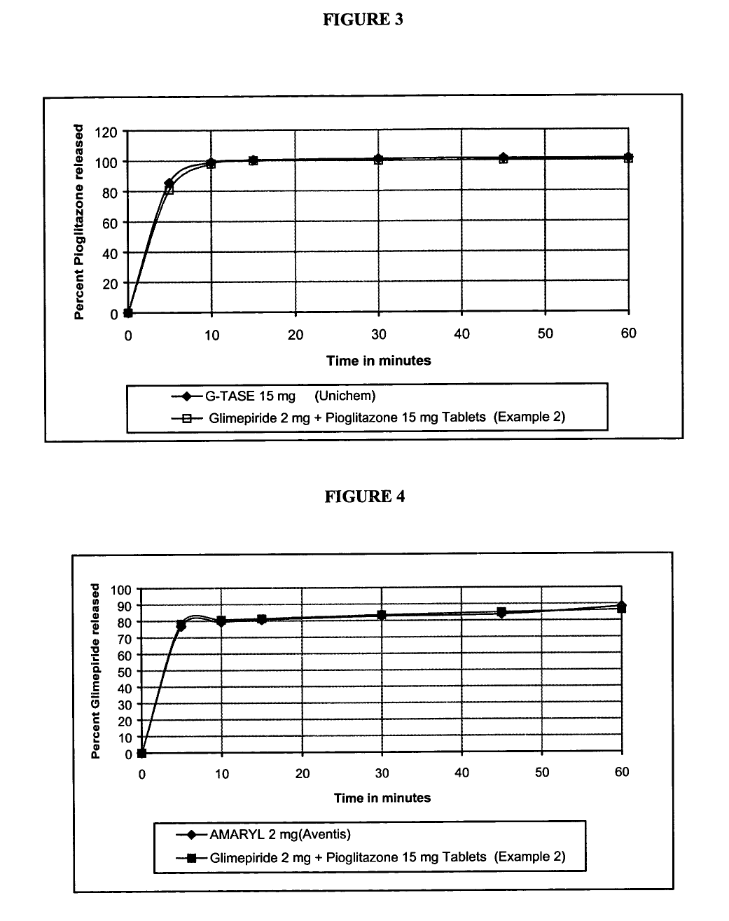

The dissolution profile for pioglitazone and glimepiride from the unit-dose combination composition of Example 2 was studied as described previously in example 1. The dissolution results for pioglitazone as recorded in Table 7 and as plotted in the profiles given in FIG. 3 reveal that pioglitazone is released from the unit-dose combination composition of Example 2 at a substantially similar rate to that of the individually marketed product.

TABLE 7

Cumulative percent pioglitazone released

| | | Glimepiride 2 mg + |
|---|---|---|
| Time | G-Tase ™ 15 mg | pioglitazone 15 mg tablets |
| (Minutes) | (Unichem) | (Example 2) |
| 5 | 85.60 | 81.54 |
| 10 | 98.91 | 97.91 |
| 15 | 100.32 | 99.57 |
| 30 | 101.11 | 100.00 |
| 45 | 101.23 | 100.58 |
| 60 | 101.23 | 100.93 |

The dissolution results for glimepiride as recorded in Table 8 and as plotted in the profiles given in FIG. 4 reveal the release of glimepiride from the unit-dose combination composition of Example 2 is at substantially the same rate as the individually marketed product.

TABLE 8

Cumulative percent glimepiride released

| | | Glimepiride 2 mg + |
|---|---|---|
| Time | Amaryl ™ 2 mg | pioglitazone 15 mg tablets |
| (Minutes) | (Aventis) | (Example 2) |
| 5 | 76.79 | 78.25 |
| 10 | 79.33 | 80.49 |
| 15 | 80.62 | 81.28 |
| 30 | 82.63 | 83.36 |
| 45 | 83.71 | 84.92 |
| 60 | 88.54 | 86.39 |

Stability of the Novel Unit-Dose Combination Composition:

The unit-dose combination composition of example 2, was also subjected to accelerated stability testing as described in example 1. The results reveal no change in the physical properties of the tablets such as color and shape. Further, results indicated no chemical degradation of the active agents as seen from the assay values at three months, which were 98.47% and 98.42% of the respective label claims of pioglitazone and glimepiride.

Thus, this example 2, which employs pioglitazone hydrochloride and higher dose of glimepiride shows that the novel unit-dose combination composition of the composition is physically and chemically stable under accelerated stability testing conditions of elevated temperature and humidity.

### EXAMPLE 3

This example illustrates the present invention in the form of unit-dose tablet containing glimepiride (1 mg) and higher dose of pioglitazone (30 mg) than was used in Examples 1 or 2, and a lower amount of diluent was used to regulate the release profiles. The pharmaceutical composition of this example is given below in Table 9.

TABLE 9

| | Weight of the |
|---|---|
| Ingredients | composition (mg) |
| Pioglitazone hydrochloride | 33.2 |
| (equivalent to pioglitazone | |
| 30 mg) | |
| Glimepiride | 1.00 |
| Microcrystalline cellulose | 15.0 |
| Lactose monohydrate | 60.0 |
| Polyvinylpyrrolidone (K-30) | 3.5 |
| Crosslinked | 5.0 |
| polyvinylpyrrolidone | |
| Purified talc | 0.5 |
| Magnesium stearate | 1.2 |
| Colloidal silicon dioxide | 0.5 |

Coating Compostion:

| | Weight of the coating |
|---|---|
| Ingredients | composition (mg) |
| Hydroxypropyl | 2.0 |
| methylcellulose | |
| Polyethylene glycol 400 | 0.3 |
| Titanium dioxide | 0.5 |
| Coloring agent (Iron | 0.5 |
| Oxide Red & Yellow) | |
| Isopropyl alcohol | q.s. |
| Methylene chloride | q.s. |

In this example the tablets were prepared and studied for content uniformity, dissolution and stability as described previously in example 1.

Characterization Studies

Content Uniformity of the Novel Unit-Dose Combination Composition:

The coated tablets prepared above were tested for the uniformity of content of pioglitazone and glimepiride. Ten (10) sample tablets (denoted below in Table 10 as CU-1 through CU-10) demonstrated an assay of glimepiride of 99.78% of label claim (1 mg per tablet) with a uniformity of content of 100.37%±3.53% and an assay of pioglitazone of 101.37% of label claim (30 mg per tablet) with a uniformity of content of 100.28% 2.73%. The content uniformity of each tablet studied in this example is given below in Table 10.

TABLE 10

| Tablets for | | |
|---|---|---|
| Content | Glimepiride | Pioglitazone |
| Uniformity | 1 mg (%) | 30 mg (%) |
| CU-1 | 103.22 | 100.41 |
| CU-2 | 99.71 | 97.88 |
| CU-3 | 106.28 | 99.18 |
| CU-4 | 97.51 | 103.61 |
| CU-5 | 98.92 | 99.49 |
| CU-6 | 102.11 | 97.27 |
| CU-7 | 95.28 | 106.31 |
| CU-8 | 96.63 | 99.67 |
| CU-9 | 104.29 | 100.28 |
| CU-10 | 99.77 | 98.66 |
| | Mean = 100.37 | Mean = 100.28 |
| | SD = 3.53 | SD = 2.73 |
| | RSD = 3.52 | RSD = 2.72 |

The data demonstrates good uniformity of content of glimepiride and for higher dose of pioglitazone hydrochloride in the tablets.

Dissolution Profile of the Novel Unit-Dose Combination Composition:

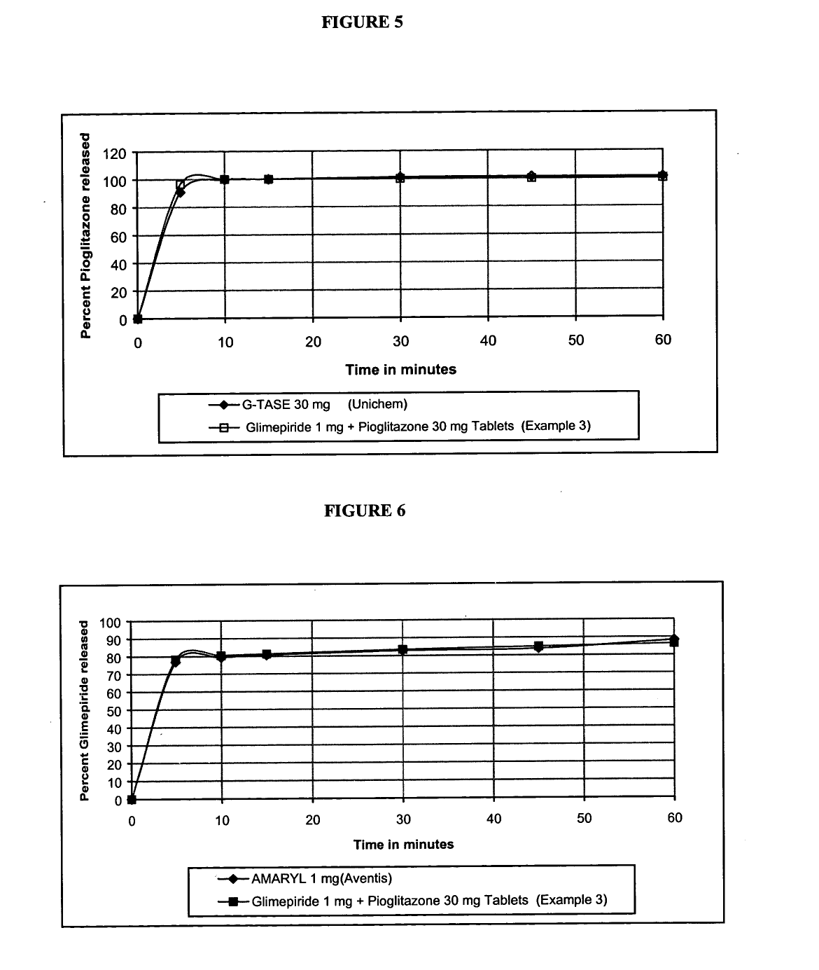

The dissolution profile for pioglitazone and glimepiride from the unit-dose combination composition of Example 3 was studied as described previously in Example 1. The dissolution results for pioglitazone as recorded in Table 11 and as plotted in the profiles given in FIG. 5 reveal that pioglitazone is released from the unit-dose combination composition of Example 3 at a substantially similar rate to that of the individually marketed product.

TABLE 11

Cumulative percent glimepiride released

| | | Glimepiride 1 mg + |
|---|---|---|
| Time | G-Tase ™ 30 mg | pioglitazone 30 mg tablets |
| (Minutes) | (Unichem) | (Example 3) |
| 5 | 90.78 | 96.55 |
| 10 | 99.80 | 100.00 |
| 15 | 100.00 | 100.00 |
| 30 | 101.20 | 100.00 |
| 45 | 101.34 | 100.08 |
| 60 | 101.41 | 100.23 |

The dissolution results for glimepiride as recorded in Table 12 and as plotted in the profiles given in FIG. 6 reveal that the release of glimepiride from the unit-dose combination composition of Example 3 is at substantially the same rate as the individually marketed product.

TABLE 12

Cumulative percent glimepiride released

| | | Glimepiride 1 mg + |
|---|---|---|
| Time | Amaryl ™ 1 mg | pioglitazone 30 mg tablets |
| (Minutes) | (Aventis) | (Example 3) |
| 5 | 76.82 | 78.87 |
| 10 | 79.33 | 80.18 |
| 15 | 80.62 | 81.54 |
| 30 | 82.78 | 83.11 |
| 45 | 83.71 | 84.47 |
| 60 | 88.29 | 86.19 |

Stability of the Novel Unit-Dose Combination Composition:

The unit-dose combination composition of Example 3 was subjected to accelerated stability testing as described previously in example 1. The results reveal no change in the physical properties of the tablets such as color and shape. Further, results indicated no chemical degradation of the active agents as seen from the assay values at three months which were 99.84% and 98.04% of the respective label claims of pioglitazone and glimepiride.

Thus, Example 3, which employs glimepiride and higher dose of pioglitazone hydrochloride than was used in Examples 1 or 2, with lower amounts of diluent, shows that the combination composition of this example is physically and chemically stable under accelerated stability testing conditions of elevated temperature and humidity.

### EXAMPLE 4

This example illustrates the present invention in the form of unit-dose tablet wherein higher dose of both glimepiride (2 mg) and pioglitazone (30 mg) was used. The pharmaceutical composition of this example is given below in Table 13.

TABLE 13

| | Weight of the |
|---|---|
| Ingredients | composition (mg) |
| Pioglitazone hydrochloride | 33.2 |
| (equivalent to pioglitazone 30 mg) | |
| Glimepiride | 2.00 |
| Microcrystalline cellulose | 15.0 |
| Lactose monohydrate | 60.0 |
| Polyvinylpyrrolidone (K-30) | 3.5 |
| Crosslinked | 5.0 |
| polyvinylpyrrolidone | |
| Purified talc | 0.5 |
| Magnesium stearate | 1.2 |
| Colloidal silicon dioxide | 0.5 |

Coating composition:

| | Weight of the coating |
|---|---|
| Ingredients | composition (mg) |
| Hydroxypropyl | 2.0 |
| methylcellulose | |
| Polyethylene glycol 400 | 0.3 |
| Titanium dioxide | 0.5 |
| Coloring agent (FD&C | 0.5 |
| Yellow No. 5) | |
| Isopropyl alcohol | q.s. |
| Methylene chloride | q.s. |

In this example the tablets were prepared and studied for content uniformity, dissolution and stability as described previously in example 1.

Characterization Studies

Content Uniformity of the Novel Unit-Dose Combination Composition:

The coated tablets of Example 4 were tested for the uniformity of content of pioglitazone and glimepiride. Ten (10) sample tablets (denoted below in Table 14 as CU-1 through CU-10) demonstrated an assay of glimepiride of 100.32% of label claim (2 mg per tablet) with a uniformity of content of 101.23%±2.94% and an assay of pioglitazone of 100.68% of label claim (30 mg per tablet) with a uniformity of content of 100.95%+3.00%. The content uniformity of each tablet studied in this example is given below in Table 14.

TABLE 14

| Tablets for Content | Glimepiride 2 mg | |
|---|---|---|
| Uniformity | (%) | Pioglitazone 30 mg (%) |
| CU-1 | 99.27 | 98.51 |
| CU-2 | 106.82 | 103.52 |
| CU-3 | 102.31 | 98.39 |
| CU-4 | 98.71 | 100.03 |
| CU-5 | 99.61 | 106.26 |
| CU-6 | 100.81 | 99.87 |
| CU-7 | 97.49 | 105.22 |
| CU-8 | 103.72 | 97.63 |
| CU-9 | 99.37 | 101.19 |
| CU-10 | 104.21 | 98.92 |
| | Mean = 101.23 | Mean = 100.95 |
| | SD = 2.94 | SD = 3.03 |
| | RSD = 2.91 | RSD = 3.00 |

The data demonstrates good uniformity of content for higher doses of glimepiride and pioglitazone hydrochloride in the tablets.

Dissolution Profile of the Novel Unit-Dose Combination Composition:

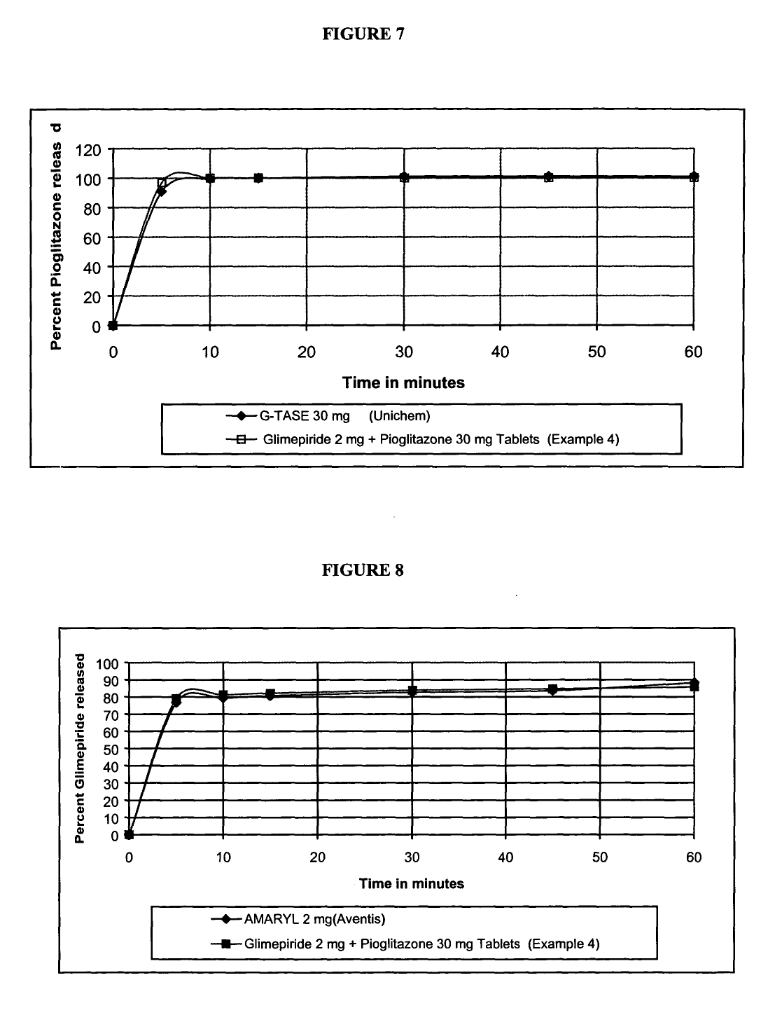

The dissolution profile for pioglitazone and glimepiride from the unit-dose combination composition of Example 4 was studied as described previously in Example 1. The dissolution results for pioglitazone as recorded in Table 15 and as plotted in the profiles given in FIG. 7 reveal that pioglitazone is released from the unit-dose combination composition of Example 4 at a substantially similar rate to that of the individually marketed product.

TABLE 15

Cumulative percent piloglitazone released

| | | Glimepiride 2 mg + |
|---|---|---|
| Time | G-Tase ™ 30 mg | pioglitazone 30 mg tablets |
| (Minutes) | (Unichem) | (Example 4) |
| 5 | 90.78 | 96.87 |
| 10 | 99.80 | 100.04 |
| 15 | 100.00 | 100.09 |
| 30 | 101.20 | 100.11 |
| 45 | 101.34 | 100.18 |
| 60 | 101.41 | 100.34 |

The dissolution results for glimepiride as recorded in Table 16 and as plotted in the profiles given in FIG. 8 reveal the release of glimepiride from the unit-dose combination composition of Example 4 at substantially the same rate as the individually marketed product.

TABLE 16

Cumulative percent glimepiride released

| | | Glimepiride 2 mg + |
|---|---|---|
| Time | Amaryl ™ 2 mg | pioglitazone 30 mg tablets |
| (Minutes) | (Aventis) | (Example 4) |
| 5 | 76.79 | 79.14 |
| 10 | 79.33 | 81.17 |
| 15 | 80.62 | 82.14 |
| 30 | 82.63 | 83.91 |
| 45 | 83.71 | 84.77 |
| 60 | 88.54 | 85.89 |

Stability of the Novel Unit-Dose Combination Composition:

The unit-dose combination composition of example 4 was subjected to accelerated stability testing as described previously in Example 1. The results reveal no change in the physical properties of the tablets such as color and shape. Furthermore, results indicated no chemical degradation of the active agents as seen from the assay values at three months which were 99.32% and 98.83% of the respective label claims of pioglitazone and glimepiride.

Thus, Example 4, which employs higher doses of glimepiride and pioglitazone hydrochloride shows that the novel unit-dose combination composition of the present invention is physically and chemically stable under accelerated stability testing conditions of elevated temperature and humidity.

What is claimed is:

1. A pharmaceutical composition for oral administration comprising:
a pharmaceutically effective amount of glimepiride; and
a pharmaceutically effective amount of a thiazolidinedione or a pharmaceutically acceptable salt of the thiazolidinedione;
wherein the composition provides a simultaneous release of the glimepiride and the thiazolidinedione or the pharmaceutically acceptable salt of the thiazolidinedione, wherein the glimepiride is released at a rate substantially similar to that obtained with an individual administration of an immediate-release dosage form of the glimepiride, and the thiazolidinedione or the pharmaceutically acceptable salt of the thiazolidinedione is released at a rate substantially similar to that obtained with an individual administration of an immediate-release dosage form of the thiazolidinedione or the pharmaceutically acceptable salt of the thiazolidinedione.

2. The composition of claim 1, wherein the glimepiride has a mean particle size of less than about 30 microns and a particle size distribution such that at least 90% of glimepiride particles are less than about 75 microns.

3. The composition of claim 1, wherein the glimepiride has a mean particle size of less than about 25 microns and a particle size distribution such that at least 90% of glimepiride particles are less than about 60 microns.

4. The composition of claim 1, wherein the glimepiride comprises about 0.2% to about 8% by weight of the composition.

5. The composition of claim 4, wherein the glimepiride comprises about 0.5% to about 3.5% by weight of the composition.

6. The composition of claim 1, wherein the thiazolidinedione comprises a member selected from the group consisting of pioglitazone and rosiglitazone.

7. The composition of claim 6, wherein the thiazolidinedione comprises pioglitazone.

8. The composition of claim 1, wherein the thiazolidinedione or the pharmaceutically acceptable salt of the thiazolidinedione comprises about 0.5% to about 45% by weight of the composition.

9. The composition of claim 7, wherein the thiazolidinedione or the pharmaceutically acceptable salt of the thiazolidinedione comprises about 4% to about 45% by weight of the composition.

10. The composition of claim 8, wherein the thiazolidinedione comprises rosiglitazone, and the thiazolidinedione or the pharmaceutically acceptable salt of the thiazolidinedione comprises about 0.5% to about 10% by weight of the composition.

11. The composition of claim 1, further comprising:
a binding agent, a diluent, a disintegrant, a glidant, a wetting agent, a lubricant, a colorant or a mixture thereof.

12. The composition of claim 1, wherein the composition is in a physical form selected from the group consisting of a pellet, a bead, a granule, a tablet and a capsule.

13. The composition of claim 12, wherein the physical form is a tablet, and the tablet includes a coating comprising a fast-dissolving film of a water-soluble polymer.

14. A pharmaceutical composition for oral administration comprising:
a pharmaceutically effective amount of glimepiride; and
a pharmaceutically effective amount of a thiazolidinedione or a pharmaceutically acceptable salt of the thiazolidinedione;
wherein the glimepiride has a mean particle size of less than about 30 microns and a particle size distribution such that at least 90% of glimepiride particles are less than about 75 microns; and
wherein the composition provides a simultaneous release of the glimepiride and the thiazolidinedione or the pharmaceutically acceptable salt of the thiazolidinedione, wherein the glimepiride is released at a rate substantially similar to that obtained with an individual administration of an immediate-release dosage form of the glimepiride, and the thiazolidinedione or the pharmaceutically acceptable salt of the thiazolidinedione is released at a rate substantially similar to that obtained with an individual administration of an immediate-release dosage form of the thiazolidinedione or the pharmaceutically acceptable salt of the thiazolidinedione.

15. A composition comprising:
a pharmaceutically effective amount of glimepiride; and
a pharmaceutically effective amount of a thiazolidinedione or a pharmaceutically acceptable salt of the thiazolidinedione;
wherein the glimepiride has a mean particle size of less than about 30 microns and a particle size distribution such that at least 90% of glimepiride particles are less than about 75 microns.

16. The composition of claim 15, wherein the mean particle size is less than about 25 microns and the particle size distribution is such that at least 90% of glimepiride particles are less than about 60 microns.

17. The composition of claim 15, wherein the glimepiride comprises about 0.2% to about 8% by weight of the composition.

18. The composition of claim 15, wherein the thiazolidinedione comprises a member selected from the group consisting of pioglitazone and rosiglitazone.

19. The composition of claim 15, wherein the thiazolidinedione or the pharmaceutically acceptable salt of the thiazolidinedione comprises about 0.5% to about 45% by weight of the composition.

20. The composition of claim 15, further comprising:
a binding agent, a diluent, a disintegrant, a glidant, a wetting agent, a lubricant, a colorant or a mixture thereof.

21. The composition of claim 15, wherein the composition is in a physical form selected from the group consisting of a pellet, a bead, a granule, a tablet and a capsule.

22. The composition of claim 21, wherein the physical form is a tablet, and the tablet includes a coating comprising a fast-dissolving film of a water-soluble polymer.

23. A method of treating non-insulin dependent diabetes mellitus or diabetes-related disorders, comprising:
administering to a mammal in need thereof, a pharmaceutical composition comprising:
a pharmaceutically effective amount of glimepiride; and
a pharmaceutically effective amount of a thiazolidinedione or a pharmaceutically acceptable salt of the thiazolidinedione;
wherein the glimepiride has a mean particle size of less than about 30 microns and a particle size distribution such that at least 90% of glimepiride particles are less than about 75 microns; and
wherein the composition provides a simultaneous release of the glimepiride and the thiazolidinedione or the pharmaceutically acceptable salt of the thiazolidinedione, wherein the glimepiride is released at a rate substantially similar to that obtained with an individual administration of an immediate-release dosage form of the glimepiride, and the thiazolidinedione or the pharmaceutically acceptable salt of the thiazolidinedione is released at a rate substantially similar to that obtained with an individual administration of an immediate-release dosage form of the thiazolidinedione or the pharmaceutically acceptable salt of the thiazolidinedione.

24. The method of claim 23, wherein the mean particle size is less than about 25 microns and the particle size distribution is such that at least 90% of glimepiride particles are less than about 60 microns.

25. The method of claim 23, wherein the glimepiride comprises about 0.2% to about 8% by weight of the composition.

26. The method of claim 23, wherein the thiazolidinedione comprises a member selected from the group consisting of pioglitazone and rosiglitazone.

27. The method of claim 23, wherein the thiazolidinedione or the pharmaceutically acceptable salt of the thiazolidinedione comprises about 0.5% to about 45% by weight of the composition.

28. The method of claim 23, wherein the pharmaceutical composition further comprises a binding agent, a diluent, a disintegrant, a glidant, a wetting agent, a lubricant, a colorant or a mixture thereof.

29. The method of claim 23, wherein the composition is in a physical form selected from the group consisting of a pellet, a bead, a granule, a tablet and a capsule.

30. The method of claim 29, wherein the physical form is a tablet, and the tablet includes a coating comprising a fast-dissolving film of a water-soluble polymer.

31. A process for preparing a pharmaceutical composition for oral administration, the process comprising:
mixing a pharmaceutically effective amount of glimepiride with a pharmaceutically effective amount of a thiazolidinedione or a pharmaceutically acceptable salt of the thiazolidinedione;
wherein the composition provides a simultaneous release of the glimepiride and the thiazolidinedione or the pharmaceutically acceptable salt of the thiazolidinedione, wherein the glimepiride is released at a rate substantially similar to that obtained with an individual administration of an immediate-release dosage form of the glimepiride, and the thiazolidinedione or the pharmaceutically acceptable salt of the thiazolidinedione is released at a rate substantially similar to that obtained with an individual administration of an immediate-release dosage form of the thiazolidinedione or the pharmaceutically acceptable salt of the thiazolidinedione.

32. A process for preparing a composition comprising:
mixing a pharmaceutically effective amount of glimepiride with a pharmaceutically effective amount of a thiazolidinedione or a pharmaceutically acceptable salt of the thiazolidinedione;
wherein the glimepiride has a mean particle size of less than about 30 microns and a particle size distribution such that at least 90% of glimepiride particles are less than about 75 microns.

33. The process of claim 32, wherein the thiazolidinedione comprises a member selected from the group consisting of pioglitazone and rosiglitazone.
